(19) United States
(12) Patent Application Publication
(10) Pub. No.: US 2019/0216840 A1
(43) Pub. Date: Jul. 18, 2019

(54) NOVEL USE OF KIRREL2 AND KIRREL2 INHIBITOR

(71) Applicant: Genome and Company, Seongnam-si (KR)

(72) Inventors: Hansoo PARK, Gyeonggido (KR); Kyoung-Wan Yoon, Gyeonggido (KR); Jinyoung Sohn, Gyeonggido (KR); Yun Yeon Kim, Gyeonggido (KR); Suro Lee, Gyeonggido (KR); Youn Kyung Houh, Gyeonggido (KR); Joo-Yeon Chung, Gyeonggido (KR); Areum Jeong, Gyeonggido (KR)

(73) Assignee: Genome and Company, Seongnam-si (KR)

(21) Appl. No.: 16/245,436

(22) Filed: Jan. 11, 2019

Related U.S. Application Data
(60) Provisional application No. 62/616,776, filed on Jan. 12, 2018.

(51) Int. Cl. A61K 31/7088; A61P 35/04; C12Q 1/02; G01N 33/574; C12N 15/113

(52) U.S. Cl. CPC A61K 31/7088; A61P 35/04; C12N 2310/113; G01N 33/574; C12N 15/1136; C12Q 1/025

(57) ABSTRACT

The present disclosure is based on the discovery that the inhibition of KIRREL2 activity or expression significantly inhibits the development, growth, invasion, and metastasis of cancer. The present disclosure provides a pharmaceutical composition for treating or preventing cancer, comprising KIRREL2 inhibitor. In addition, the present disclosure provides a pharmaceutical composition for immune-enhancing, comprising KIRREL2 inhibitor. Furthermore, the present disclosure provides a method of screening of anti-cancer agent using KIRREL2, and a method of providing information necessary for analysis of cancer prognosis using KIRREL2.

### CROSS-REFERENCE TO THE RELATED APPLICATION

This application claims priority from U.S. Application No. 62/616,776 filed on Jan. 12, 2018, the disclosure of which is incorporated herein in its entirety by reference.

### TECHNICAL FIELD

The present disclosure provides a pharmaceutical composition for treating or preventing cancer, comprising a KIRREL2 inhibitor, and a method of treating or preventing cancer by administering a KIRREL2 inhibitor to a subject in need thereof. In addition, the present disclosure provides a pharmaceutical composition for immune-enhancing, comprising a KIRREL2 inhibitor and a method of immune-enhancing by administering a KIRREL2 inhibitor to a subject in need thereof. Furthermore, the present disclosure provides a method of screening of anti-cancer agent using KIRREL2, and a method of providing information necessary for analysis of cancer prognosis using KIRREL2.

### BACKGROUND ART

Despite advances in understanding the etiology of cancer and the methods for treating cancer over the past several years, it is still the leading cause of death worldwide. Although anti-cancer treatments exist for many malignancies, such treatments often do not fully control such malignancies or are not effective in all patients. Most of the methods currently being used to treat cancer are relatively non-selective. The affected tissue is removed through surgery, the size of solid tumors is reduced through radiation therapy, or chemotherapy is used to kill cancer cells rapidly. In particular, the chemotherapy can cause the drug resistance, and sometimes restricts the administrable dose. It causes severe side effects so that they may rule out the use of potentially effective agents. Accordingly, there is a need to develop more target-specific and effective cancer therapies.

The adaptive immune system of the human is a very precise system which is able to specifically remove cancer cells. In particular, T cells determine cell mediated adaptive immunity, and recognize and remove non-self antigens or abnormal antigens that a cell is exposed to. T cells express about 20,000 to 40,000 TCR molecules per cell, and recognize several antigens (determined by their peptide sequences) among the 100,000 pMHC molecules of APC to begin signal transfer. Such TCR molecules should function as highly sensitive sensors which need to recognize very minute changes in the antigen and transfer signals. This cell-mediated adaptive immunity operates in a very precise manner to effectively remove cancer cells. If an antigen-specific adaptive immune system does not operate normally, serious problems are caused in the ability to remove cancer cells. For example, if the protein PD-L1 or PD-L2 on the surface of a cancer cell binds to the protein PD-1 on the surface of a T cell, the T cell is not able to attack cancer cells. Therefore, for effective cancer treatment, it is necessary to remove the factors that hinder T cell's ability to remove cancer cells.

Accordingly, the inventors have conducted research to develop a method of cancer treatment using the human immune system, and identified that inhibition of the activity and expression of KIRREL2 leads to substantial suppression of development, growth, invasion and metastasis of cancer.

### Technical Problem

One purpose of the present disclosure is to provide a pharmaceutical composition for treating or preventing cancer.

Another purpose of the present disclosure is to provide a pharmaceutical composition for immune-enhancing.

Another purpose of the present disclosure is to provide a method of screening of anti-cancer agent.

Another purpose of the present disclosure is to provide a method of providing information necessary for analysis of cancer prognosis.

### Technical Solution

To achieve the purposes of the present disclosure, one aspect of the present disclosure provides a pharmaceutical composition for treating or preventing cancer, comprising KIRREL2 inhibitor as an active ingredient and a method of treating or preventing cancer by administering a KIRREL2 inhibitor to a subject in need thereof.

The term “KIRREL2 (Kin of IRRE-like protein 2)” refers to a protein that is encoded by KIRREL2 gene, which belongs to the type I transmembrane proteins and the immunoglobulin superfamily of cell adhesion molecules. It is reported to be usually located at the adherent junctions of pancreatic beta cells, and to regulate insulin secretion. The KIRREL2 is a member of the NEPH protein family, and is also known as ‘NEPH3’.

The KIRREL2 may be human-derived KIRREL2. More specifically, the amino acid sequence of KIRREL2 may comprise the sequence of NCBI Reference Sequence: NP_954649.3 disclosed in the NCBI. The amino acid sequence of KIRREL2 may be, but is not limited to, amino acid sequences having at least 80%, 85%, 90% or 95% identity with the sequence of NCBI Reference Sequence: NP_954649.3, as well as amino acid sequences having the property or function of KIRREL2.

The gene of KIRREL2 may include a nucleic acid sequence encoding the amino acid sequence of human-derived KIRREL2, or the nucleic acid sequence of NCBI Reference Sequence: NM_199180.3 disclosed in the NCBI. The nucleic acid sequence of KIRREL2 may be, but are not limited to, nucleic acid sequences having at least 80%, 85%, 90% or 95% identity with the sequence of NCBI Reference Sequence: NM_199180.3, as well as nucleic acid sequences that can produce amino acids having the property or function of KIRREL2.

The term “KIRREL2 inhibitor” refers to substances that inhibit the activity or expression of KIRREL2. The KIRREL2 inhibitor can preferably suppress the function of cancer cells evading T cells. The KIRREL2 inhibitor blocks the activity of KIRREL2 existing in a cancer cell, thereby suppressing the mechanism that T cells are rendered unable to attack cancer cells by KIRREL2 and maintaining the immune activity of T cells against cancer cells. Alternatively, the KIRREL2 inhibitor specifically binds to the KIRREL2 protein, and interferes with binding of KIRREL2 to T cells. Alternatively, the KIRREL2 inhibitor suppresses a particular metabolic pathway of KIRREL2 to reduce the expression of protein, or causes KIRREL2 to denature so that the protein loses its activity. Therefore, the KIRREL2 inhibitor according to the present disclosure is very effective in treating or preventing cancer. The KIRREL2 inhibitor may include, but are not limited to, any compounds, proteins, fusion proteins, antibodies, amino acids, peptides, viruses, carbohydrates, lipids, nucleic acids, extracts or fractions so long as it inhibits the activity or expression of KIRREL2.

In one embodiment, the KIRREL2 inhibitor is one that reduces the expression of KIRREL2 in a cancer cell compared to a cancer cell not treated with the KIRREL2 inhibitor. Reduction in expression of KIRREL2 may refer to lowered or no level of mRNA and/or protein produced from the KIRREL2 gene. The KIRREL2 inhibitor may include, but are not limited to, antisense nucleic acid, siRNA, shRNA, miRNA, ribozyme, etc. which binds in a complementary manner to DNA or mRNA of the KIRREL2 gene.

The term “antisense nucleic acid” refers to DNAs or RNAs comprising nucleic acid sequences complementary to the sequence of certain mRNA, or fragments or derivatives thereof, which bind to or hybridize with the complementary sequences in mRNA and inhibit the translation of mRNA into protein.

The term “siRNA (small interfering RNA)” refers to a short double chain RNA which is able to induce the RNAi (RNA interference) through cleavage of certain mRNA. The siRNA comprises a sense RNA strand having a sequence homologous to the mRNA of the target gene, and an antisense RNA strand having a sequence complementary thereto. The siRNA can inhibit the expression of the target gene, and thus can be used in gene knockdown, genetic therapy, etc.

The term “shRNA (short hairpin RNA)” is a single strand RNA, which comprises a stem portion forming a double strand portion through hydrogen bonds, and a loop portion. It is processed by a protein such as Dicer to be converted into siRNA, and performs the same function as siRNA.

The term “miRNA (micro RNA)” refers to 21 to 23 non-coding RNAs which modulate gene expression after transcription by promoting the degradation of target RNA or by suppressing its translation.

The term “ribozyme” refers to an RNA molecule that has an enzyme-like function, recognizing a particular base sequence and cutting the same. The ribozyme comprises an area that specifically binds to a complementary base sequence of a target messenger RNA strand, and an area that cleaves the target RNA.

The antisense nucleic acid, siRNA, shRNA, miRNA, ribozyme, etc. that binds complementarily to the DNA or mRNA of KIRREL2 gene can inhibit the translation of mRNA of KIRREL2, its translocation into the cytoplasm, its maturation, or any other activities crucial for the biological functions of KIRREL2.

In one embodiment, the KIRREL2 inhibitor is one that deactivates the function of KIRREL2 or reduces the activity thereof in a cancer cell compared to a cancer cell not treated with the KIRREL2 inhibitor. The KIRREL2 inhibitor may include, but are not limited to, compounds, peptides, peptide mimetics, fusion proteins, antibodies, aptamers, etc. that bind specifically to the KIRREL2 protein.

The term “specific” or “specifically” refers to the ability to bind to only a target protein without affecting other proteins in the cell.

The term “antibody” may include monoclonal antibodies, chimera antibodies, polyclonal antibodies, humanized antibodies and human antibodies, and may also include new antibodies as well as antibodies known to the art or commercialized in the art. The antibody may include not only the forms having a full length comprising 2 heavy chains and 2 light chains but also the functional fragments of antibody molecules, so long as they specifically bind to KIRREL2. The functional fragment of antibody molecule refers to a fragment having at least its antigen-binding function, and may include, but are not limited to, Fab, F(ab′), F(ab′)2, Fv, etc.

The term “peptide mimetics” refers to a peptide or non-peptide which inhibits the binding domain of KIRREL2 protein that induces KIRREL2 activity.

The term “aptamer” refers to a single strand nucleic acid (DNA, RNA or modified nucleic acid) having in itself a stable tertiary structure and being able to bind to a target molecule with high affinity and specificity.

The substance inhibiting the activity or expression of KIRREL2 which is comprised in the pharmaceutical composition of the present disclosure can inhibit suppression of T cell function by KIRREL2, and accordingly can increase or maintain the ability of T cells to attack and kill cancer cells. Here, the ability of T cells to attack and kill cancer cells in a group treated with a KIRREL2 inhibitor may be increased by 5% to 200% as compared to a group not treated with a KIRREL2 inhibitor. Thus, the pharmaceutical composition of the present disclosure can be useful in the preventing or treating cancer.

The cancer that can be treated or prevented by the pharmaceutical composition of the present disclosure may include, but are not limited to, stomach cancer, lung cancer, liver cancer, colorectal cancer, colon cancer, small intestinal cancer, pancreatic cancer, brain cancer, bone cancer, melanoma, breast cancer, sclerosing adenosis, uterine cancer, cervical cancer, head and neck cancer, esophageal cancer, thyroid cancer, parathyroid cancer, renal cancer, sarcoma, prostate cancer, urethral cancer, bladder cancer, blood cancer, leukemia, lymphoma, fibroadenoma, etc.

The pharmaceutical composition according to the present disclosure may comprise the active ingredient alone, or may additionally comprise one or more pharmaceutically acceptable carriers, excipients, diluents, stabilizing agents, preserving agent, etc.

The Pharmaceutically acceptable carriers may include, for example, carriers for oral administration or non-oral administration. The carriers for oral administration may include, for example, lactose, starch, cellulose derivatives, magnesium stearate, stearic acid, etc. The carriers for non-oral administration may include, for example, water, suitable oils, saline, aqueous glucose, glycols, etc. The pharmaceutically acceptable stabilizing agents may include, for example, antioxidants such as sodium bisulfate, sodium sulfite or ascorbic acid. The pharmaceutically acceptable preserving agents may include, for example, benzalkonium chloride, methyl- or propyl-paraben, chlorobutanol, etc. Other pharmaceutically acceptable carriers may be those disclosed in the literature “Remington's Pharmaceutical Sciences, 19th ed., Mack Publishing Company, Easton, Pa., 1995”.

The pharmaceutical composition of the present disclosure may be administered to animals including human using various methods. For example, it may be administered orally or parenterally. The parenteral administration may include, but are not limited to, intravenous, intramuscular, intraarterial, intramarrow, intradural, percutaneous, subcutaneous, intraperitoneal, intranasal, intraintestinal, topical, sublingual, rectal administration, etc.

The pharmaceutical composition of the present disclosure may be prepared into formulations for oral or parenteral administration, depending on the administration route as described in the above.

The formulation for oral administration may be prepared in the form of powders, granules, tablets, pills, sugar-coated pills, capsules, liquids, gels, syrups, slurries, suspensions, etc., using methods known in the art. For example, the active ingredient of the present disclosure may be mixed with suitable excipient(s) and/or adjuvant(s), and then processed into a granule mixture to obtain a tablet or a sugar-coated tablet for oral administration. Examples of suitable excipients may include, but are not limited to, sugars including lactose, dextrose, sucrose, sorbitol, mannitol, xylitol, erythritol, maltitol, etc., starches including corn starch, wheat starch, rice starch, potato starch, etc., celluloses including cellulose, methyl cellulose, sodium carboxymethylcellulose, hydroxypropylmethylcellulose, etc., and fillers such as gelatin, polyvinyl pyrrolidone, etc. Optionally, disintegrating agents such as crosslinked polyvinyl pyrrolidone, agar, alginic acid or sodium alginate may be added. Further, the pharmaceutical composition of the present disclosure may further comprise anticoagulants, lubricants, wetting agents, aromatic agents, emulsifiers and preservatives, etc.

The formulation for parenteral administration may be prepared in the form of injections, gels, aerosols, nasal inhalers using methods known in the art.

These administration forms may refer to those disclosed in the literature known in the art “Remington's Pharmaceutical Science, 15th Edition, 1975. Mack Publishing Company, Easton, Pa. 18042, Chapter 87: Blaug, Seymour”.

The total effective dose of the pharmaceutical composition according to the present disclosure may be administered to a subject in a single dose, or in multiple doses through a fractionated treatment protocol.

The appropriate dose of the pharmaceutical composition according to the present disclosure or the contents of active ingredient in the pharmaceutical composition may be determined considering various factors such as administration route, times administered, patient age, body weight, health, gender, severity of disease, diet and excretion rate, etc. by a person having ordinary skill in the art. For example, the total dose of the pharmaceutical composition according to the present disclosure may be about 0.01 μg to 1,000 mg per 1 kg body weight of a patient per day, or 0.1 μg to 100 mg. There is no particular limit to the dosage form, administration route and administration method, so long as the pharmaceutical composition shows the effect of the invention.

Another aspect of the present disclosure provides a pharmaceutical composition for immune-enhancing in a subject, comprising KIRREL2 inhibitor as an active ingredient.

When the pharmaceutical composition is administered to a subject in need thereof, it can fully or partially reduce the expression or activity of KIRREL2 in the subject to increase the level of T cell-mediated immune response.

Accordingly, the pharmaceutical composition of the present disclosure can be used for immune-enhancing. For example, it can be used for the subject in need of prevention, treatment or improvement of diseases related to immunodeficiency, lower immune function, immune system damage, immunocompromising, etc.

Another aspect of the present disclosure is to provide a method of treating or preventing cancer in a subject, comprising administering to the subject a KIRREL2 inhibitor(s). And also, another aspect of the present disclosure provides a method of immune-enhancing in a subject, comprising administering to the subject a KIRREL2 inhibitor(s). In these methods, unless specifically mentioned otherwise, the terms associated have the same meaning as the terms explained for the pharmaceutical compositions in the above.

Another aspect of the present disclosure is to provide a method of screening an anti-cancer agent comprising:
(a) treating a cancer cell with a candidate of anti-cancer agent; and
(b) measuring the expression or activity of KIRREL2 in the cancer cell.

Optionally, the method of screening an anti-cancer agent may further comprise a step of determining the candidate anti-cancer agent to be the anti-cancer agent if a group treated with the candidate anti-cancer agent shows a lower (or significantly lower) level of expression of KIRREL2 mRNA or protein or a lower (or significantly lower) level of suppression of T cell activity by KIRREL2 compared to a group not treated with the candidate anti-cancer agent. Here, the lower (or significantly lower) level may indicate an amount decreased by 5% to 95% (e.g., 10%, 15%, 20%, 25%, 30%, 35%, 40%, 45%, 50%, 55%, 60%, 65%, 70%, 75%, 80%, 85% and 90%). The group not treated with the candidate anti-cancer agent may be cancer cells to which no substance is added, or to which any substance such as anti-cancer agent other than KIRREL2 inhibitors is treated.

The term “screening” refers to finding the target materials having the particular properties such as sensitivity or activity among proteins, fusion proteins, antibodies, peptides, antibiotics, enzymes, compounds or any other substances.

The term “candidate anti-cancer agent” may refer to a nucleic acid, protein, antibody, compound, extract or natural substance that is randomly selected or is thought to be able to inhibit the expression or activity of KIRREL2 according to the usual selection method. The candidate of anti-cancer agent may preferably be a substance that inhibits the expression and/or activity of KIRREL2.

The expression or activity of KIRREL2 may be measured by determining the level of expression of the mRNA or protein of KIRREL2, or by determining the degree to which T cell activity is suppressed by KIRREL2.

The method of determining the level of expression of the mRNA of KIRREL2 may include, but are not limited to, any method conventionally known to the art such as reverse transcriptase PCR, competitive reverse transcriptase PCR, real-time reverse transcriptase PCR, RNase protection assay, Northern blotting, DNA chip or RNA chip.

The method of determining the level of expression of the KIRREL2 protein may include, but are not limited to, any method conventionally known to the art such as Western blot, ELISA, radioimmunoassay analysis, radial immunodiffusion, Ouchterlony immunodiffusion, rocket immunoelectrophoresis, tissue immunohistochemistry, immunoprecipitation assay, complement fixation assay, FACS or protein chip.

The method of determining the degree of T cell activity inhibition by KIRREL2 may include, but are not limited to, any method conventionally known to the art such as RT-PCR, Western Blot, ELISA, radioimmunoassay, radioimmunodiffusion, Ouchterlony immunodiffusion, rocket immunoelectrophoresis, immunohistochemistry, immunoprecipitation, complete fixation assay, or FACS.

In addition, in the method of screening of the present disclosure, confirmation of KIRREL2 activity inhibition may be performed using conventional methods such as reacting the KIRREL2 protein and a candidate substance to measure an activity, yeast two-hybrid, searching for phage-displayed peptide clones binding to KIRREL2 protein, HTS (high throughput screening) using natural material and chemical libraries, drug hit HTS, cell-based screening, or DNA array-based screening.

The method of screening an anti-cancer agent may be performed either in vitro or in vivo. For in vivo, the step of treating a cancer cell with a candidate of anti-cancer agent may be substituted by a step of administering a candidate of anti-cancer agent to a subject having cancer cells or suffering from cancer. Such a subject may be am animal such as human, mouse, etc.

The method of screening an anti-cancer agent is based on the novel disclosure in the present invention that inhibition of the activity or expression of KIRREL2 can suppress the function of cancer cells evading T cells. The method of screening of the present disclosure is very advantageous in that it allows for easy development of new anti-cancer agents through a simple and inexpensive method.

Another aspect of the present disclosure provides a method of providing information necessary for analysis of cancer prognosis, comprising measuring expression or activity of KIRREL2 in cells or tissues isolated from a subject.

In the method, the terms associated with the expression or activity of KIRREL2 and its measurement, unless specifically mentioned otherwise, have the same meaning as the terms explained for the composition and the screening method.

The term “prognosis” refers to predictions as to progress of disease, improvement of disease, recurrence of disease, metastasis, and likelihood of death. For example, in the present disclosure, the prognosis refers to the possibility of curing a cancer patient or improving the condition of cancer patient.

The cell or tissue isolated from the subject may be a cancer cell or a tissue wherein cancer have occurred or cancer cells exist.

The method of providing information necessary for analysis of cancer prognosis is based on the fact that the lower activity or expression of KIRREL2 in cancer cells can increase T cell activity and proliferation, thereby increasing cancer treatment effect.

The articles “a” and “an” are used herein to refer to one or to more than one (i.e. to at least one) of the grammatical object of the article. By way of example, “an ingredient” means one ingredient or more than one ingredient.

### BRIEF DESCRIPTION OF FIGURES

FIG. 1 shows the proliferation (%) of CD4+ T cells suppressed by KIRREL2.

FIG. 2 shows the proliferation (%) of CD8+ T cells suppressed by KIRREL2.

FIGS. 3A, 3B, 3C and 3D show the cytotoxicity (%) of PBMC when lung cancer cell line H1129 and PBMC were treated with KIRREL2 inhibitors.

FIGS. 4A, 4B, 4C and 4D show the cytotoxicity (%) of PBMC when colon cancer cell line HCT-116 and PBMC were treated with KIRREL2 inhibitors.

FIGS. 5A, 5B, 5C and 5D show the cytotoxicity (%) of PBMC when breast cancer cell line MDA-MB-231 and PBMC were treated with KIRREL2 inhibitors.

FIGS. 6A, 6B, 6C and 6D show the cytotoxicity (%) of PBMC when gastric cancer cell line MKN-74 and PBMC were treated with KIRREL2 inhibitors.

FIGS. 7A, 7B, 7C and 7D show the cytotoxicity (%) of PBMC when leukemia cell line U937 and PBMC were treated with KIRREL2 inhibitors.

FIGS. 8A and 8B show the change of tumor size in mouse treated with KIRREL2 inhibitors.

### EXAMPLES

In the following, exemplary embodiments of the inventive concept will be explained in further detail with reference to examples. However, the following examples are meant to exemplify the present invention, and the scope of the invention is not restricted by these examples.

### Example 1. Inhibition of KIRREL2 on T Cells Activity

This example is to confirm whether KIRREL2 suppresses the proliferation and activity of the T cell, and ensures that cancer cells evade the T cell-mediated immune system.

### 1.1. Preparation of CD4+ Cells and CD8+ T Cells

Human blood was placed in a 10 ml tube coated with EDTA (or heparin) and mixed with PBS at a ratio of 1:1. Ficoll-Paque PLUS was placed in a 50 ml tube, and then the blood sample was added. After centrifugation, human PBMCs (peripheral blood mononuclear cells) were collected. The resultant was centrifuged, and the supernatant was removed. Then, RBC lysis (1×) was added, pipetted, and stored on ice for 3 minutes. After that, 50 ml of 10% FBS RPMI1640 was added, and the mixture was centrifuged to remove the supernatant. Then, FACS buffer was added, and the supernatant was removed by centrifugation. Subsequently, 50 ml of MACS buffer (PBS containing 0.5% bovine serum albumin and 2 mM EDTA) was added, the number of cells was counted, and the supernatant was completely removed after centrifugation.

CD4+ T cells and CD8+ T cells were resuspended in 40 μl MACS buffer at 1×10⁷ cells, and placed in tubes and stored in the refrigerator for 5 minutes. Subsequently, 30 μl of MACS buffer based on the number of 1×10⁷ cells was added to the resultant, and 20 μl of anti-biotin microbeads were added and mixed. Then, CD4+ T cells and CD8+ T cells were separated using LS column, and were counted.

The prepared CD4+ T cells and CD8+ T cells were mixed with 1 μl of CFSE (carboxyfluorescein succinimidyl ester) based on the number of 2×10⁶ cells, and stored at 37° C. for 3 minutes. Then, FBS was added into tubes containing the CD4+ T cells and CD8+ T cells respectively, and stored on ice 10 minutes. Thereafter, the supernatant was removed by centrifugation. The resultant was added with 30 ml of FACS buffer, pipetted, and centrifuged to remove the supernatant. Then, the resultant was mixed with 10 ml of 10% FBS RPMI1640, and the number of cells was counted.

### 1.2. Measurement of T Cell Activity

The recombinant human IgG1 Fc protein (Cat. No. 110-HG) and the recombinant human PD-L1/B7-H1 Fc chimera protein (Cat. No. 156-B7) were purchased from R&D systems, and the recombinant human KIRREL2 Fc Tag protein (Cat. No. 15674-H02H) was purchased from Sino Biological.

10 μg/ml of each protein was mixed with 1.0 μg/ml, 2.0 μg/ml, 4.0 μg/ml, or 6.0 μg/ml of anti-CD3 antibody (BioLegend, Cat. No. 317325) in PBS, respectively. The resultant mixture was coated on 96-well plates at 4° C., and the wells were washed three times with PBS.

The CD4+ T cells and CD8+ T cells prepared in the Example 1.1 were added to each well of the 96-well plate at the number of 2×10⁶ cells in an amount of 200 μl, and then incubated.

CD4+ T cells and CD8+ T cells were activated by anti-CD3 antibody for 72 hours. The proliferation of CD4+ T cells and CD8+ T cells can be confirmed by the degree of CFSE fluorescent cell staining, and was analyzed by flow cytometry using FACSDiVa software (BD Biosciences).

### 1.3. Results

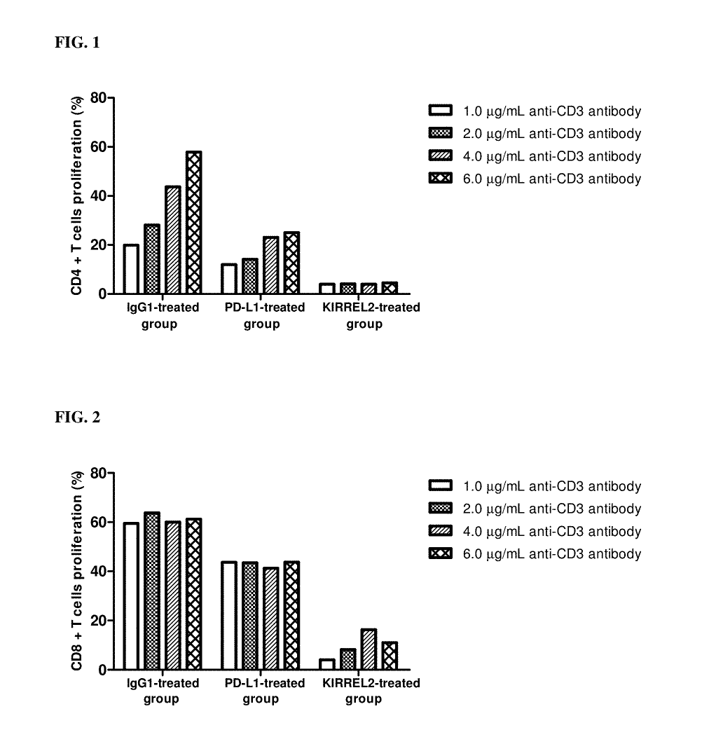

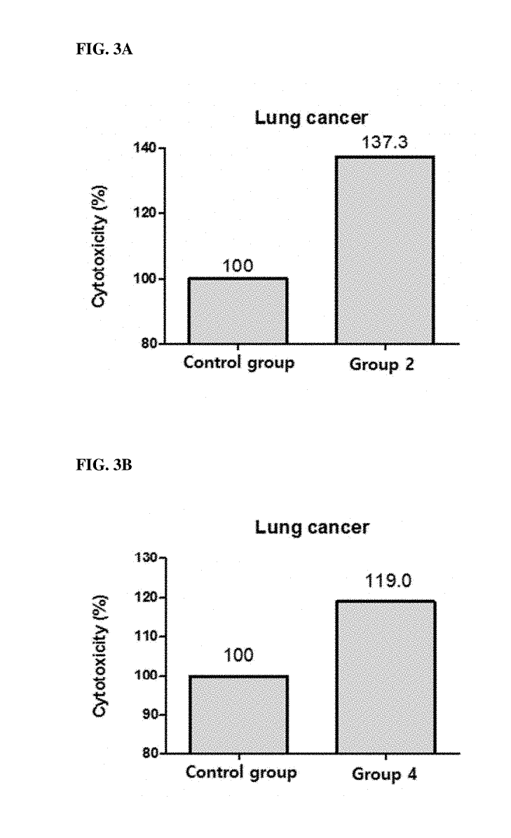

FIG. 1 and FIG. 2 show the percent proliferation (%) of CD4+ T cells and CD8+ T cells, respectively.

The control group treated with PD-L1 inhibited the proliferation of both CD4+ T cells and CD8+ T cells compared to the control group treated with IgG1. The PD-L1 binds to PD-1, a protein on the surface of T cells, and inhibits the proliferation of T cells. Accordingly, it results in suppressing the function of T cells attacking and killing cancer cells.

The group treated with KIRREL2 remarkably inhibited the proliferation of both CD4+ T cells and CD8+ T cells compared to the control group treated with IgG1 and the control group treated with PD-L1. It means that KIRREL2 suppresses the proliferation of T cells much more than PD-L1. Thus, if KIRREL2 is neutralized by blocking or knockdown, it can result in suppressing the T cell proliferation inhibition of KIRREL2. Accordingly, the cancer treatment can be effectively achieved.

### Example 2. PBMC Cytotoxic Function Assay

This example is to confirm whether the cytotoxic ability of PBMC against cancer cells is increased when KIRREL2 is neutralized using KIRREL2 inhibitors.

### 2.1. Preparation of PBMC

Human blood was placed in a 10 ml tube coated with EDTA (or heparin) and mixed with PBS at a ratio of 1:1. Ficoll-Paque PLUS was placed in a 50 ml tube, and then the blood sample was added. After centrifugation, human PBMCs were collected. The resultant was centrifuged, and the supernatant was removed. Then, RBC lysis (lx) was added, pipetted, and stored on ice for 3 minutes. After that, 50 ml of 10% FBS RPMI1640 was added, and the mixture was centrifuged to remove the supernatant. Then, FACS buffer was added, and the supernatant was removed by centrifugation. Subsequently, 50 ml of MACS buffer (PBS containing 0.5% bovine serum albumin and 2 mM EDTA) was added, the number of cells was counted, and the supernatant was completely removed after centrifugation.

96-well plates were coated with 1.0 μg/ml of anti-CD3 antibody (BioLegend, Cat. No. 317325) in PBS at 4° C., and the wells were washed three times with PBS. The PBMC prepared in the above was mixed with 10% FBS RPMI1640, and was added to each well of the 96-well plate at the number of 6×10⁵ cells in an amount of 100 μl. The PBMC was activated by anti-CD3 antibody for 72 hours.

### 2.2. Preparation of Cancer Cells

Lung cancer cell line H1129, colon cancer cell line HCT-116, breast cancer cell line MDA-MB-231, gastric cancer cell line MKN-74, and leukemia cell line U937 were respectively mixed with 1 μl of CFSE (carboxyfluorescein succinimidyl ester), and then stored at 37° C. for 3 minutes. Subsequently, FBS was added into tubes containing cancer cells and stored on ice for 10 minutes. Thereafter, the supernatant was removed by centrifugation. The resultant was added with 30 ml of FACS buffer, pipetted, and centrifuged to remove the supernatant. Then, 10% FBS RPMI1640 was added, pipetted, and centrifuged to remove the supernatant. Thereafter, the resultant was mixed with 10 ml of 10% FBS RPMI1640, and the number of cells was counted.

Each PBMC-containing well of the 96-well plate prepared in the Example 2.1 was added with the cancer cells at the number of 3×10⁴ cells in an amount of 100 μl.

### 2.3. Measurement of Cytotoxicity of PBMC Against Cancer Cells

The mixtures of PBMCs and cancer cells were prepared in the Example 2.2. These mixtures were incubated with 10 μg/mL of anti-human KIRREL2 antibody or 50 nM of human KIRREL2 siRNA for 24 hours.

Table 1 below provides the non-treated control group and Groups 1 to 6 using six neutralizing antibodies for blocking KIRREL2.

TABLE 1

| | Human KIRREL2 neutralizing antibody |
|---|---|
| Control group | Not treated |
| Group 1 | anti-human KIRREL2 antibody (R&D, MAB2564) |
| Group 2 | anti-human KIRREL2 antibody (Bioss, bs6721R) |
| Group 3 | anti-human KIRREL2 antibody (Genetex, GTX45930) |
| Group 4 | anti-human KIRREL2 antibody (Novusbio, NBP59231) |
| Group 5 | anti-human KIRREL2 antibody (R&D, AF2564) |
| Group 6 | anti-human KIRREL2 antibody (Thermo, PA5-69662) |

In addition, Table 2 below provides the non-treated control group and Groups 7 to 9 using three siRNAs for knockdown of KIRREL2.

TABLE 2

| | Human KIRREL2 siRNA |
|---|---|
| Control | Not treated |
| group | |
| Group 7 | Sense (5′-CGUGUGACAUCUUUCCAAUtt-3′) |
| | (SEQ ID NO: 1) |
| | Antisense (5′-AUUGGAAAGAUGUCACACGtt-3′) |
| | (SEQ ID NO: 2) |
| Group 8 | Sense (5′-CCAACCAACGGUUACUACAtt-3′) |
| | (SEQ ID NO: 3) |
| | Antisense (5′-UGUAGUAACCGUUGGUUGGgt-3′) |
| | (SEQ ID NO: 4) |
| Group 9 | Sense (5′-GAGAGCACCUUAACCCUGAtt-3′) |
| | (SEQ ID NO: 5) |
| | Antisense (5′-UCAGGGUUAAGGUGCUCUCca-3′) |
| | (SEQ ID NO: 6) |

After three days from incubating the mixtures of PBMCs and cancer cells with anti-KIRREL2 antibody or KIRREL2 siRNA, cells were stained with 7-aminoactinomycin D (7-AAD; BD Pharmingen, San Diego, Calif., USA) to detect lysed cells. The cytotoxicity of PBMC against cancer cells was analyzed by determining FL-1 (CFSE) and FL-3 (7-AAD) staining using a FACSDiVa software (BD Biosciences).

### 2.4. Results

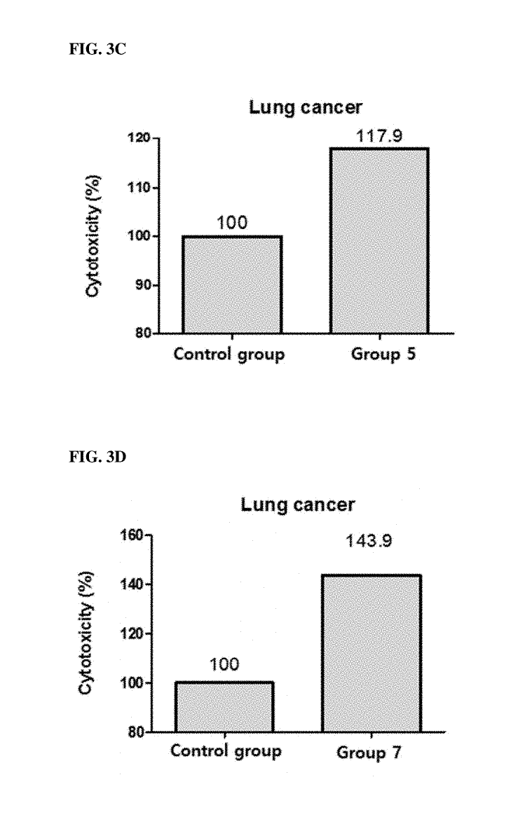

FIGS. 3A, 3B, 3C and 3D provide the results on the lung cancer cell line H1129. As shown in FIGS. 3A, 3B and 3C, when the lung cancer cell line H1129 and PBMC were treated with KIRREL2 neutralizing antibodies, the cytotoxicity against lung cancer cell was significantly increased compared to the non-treated control group even though there is more or less degree of difference depending on the type of antibody. Further, as shown in FIG. 3D, the cytotoxicity against lung cancer cell was also significantly increased when it was treated with KIRREL2 siRNA.

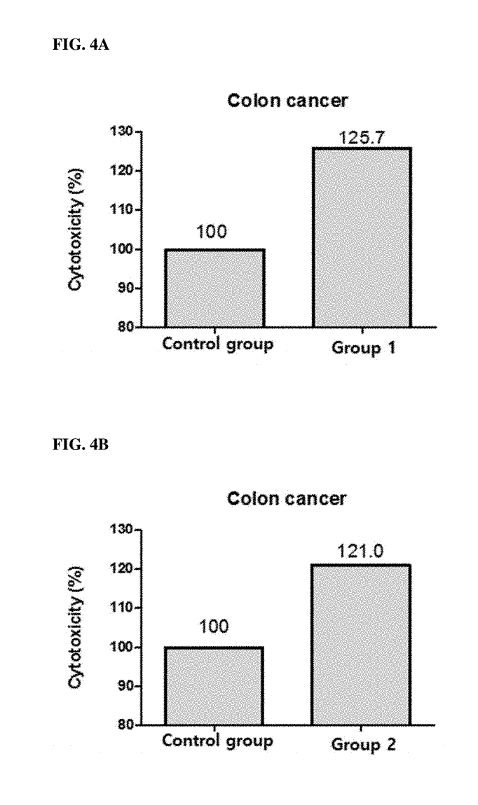

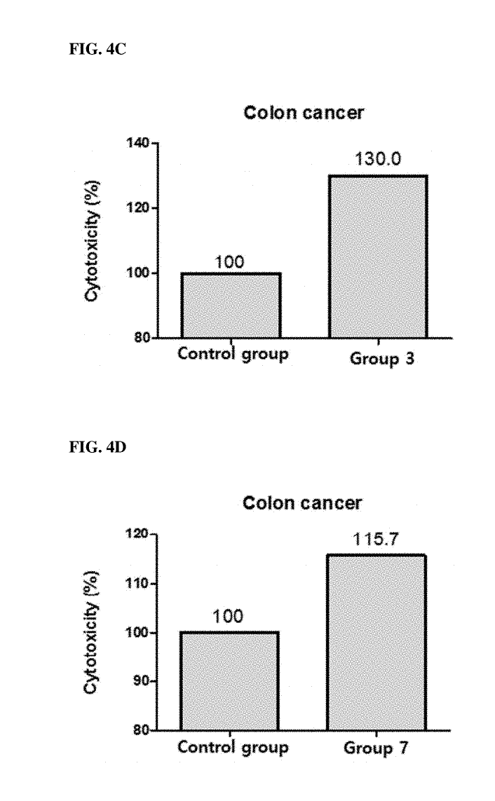

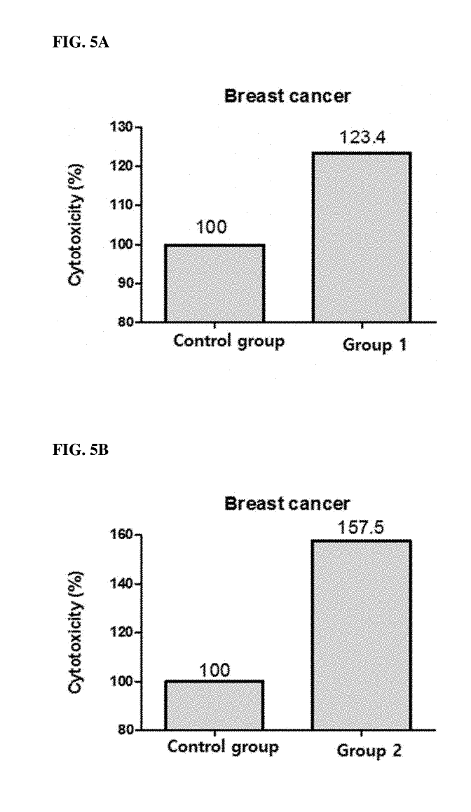

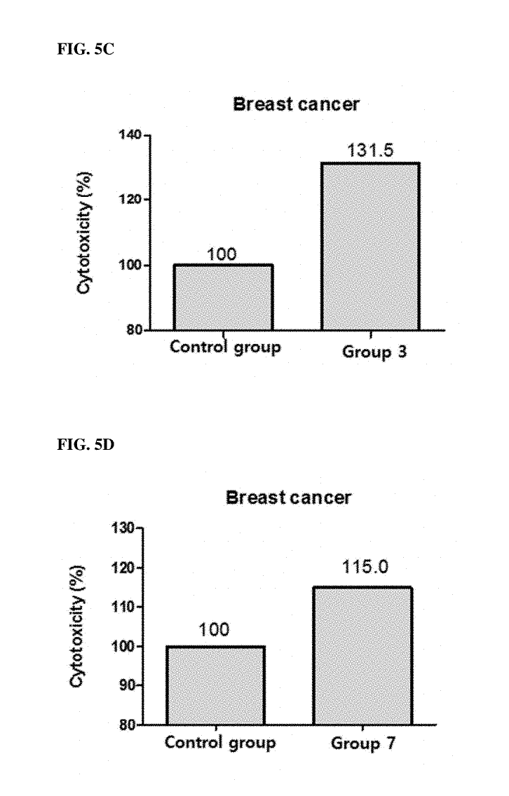

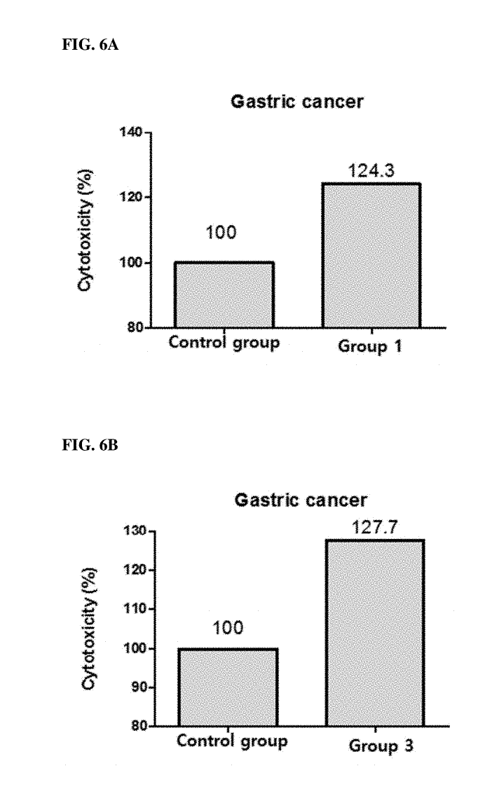

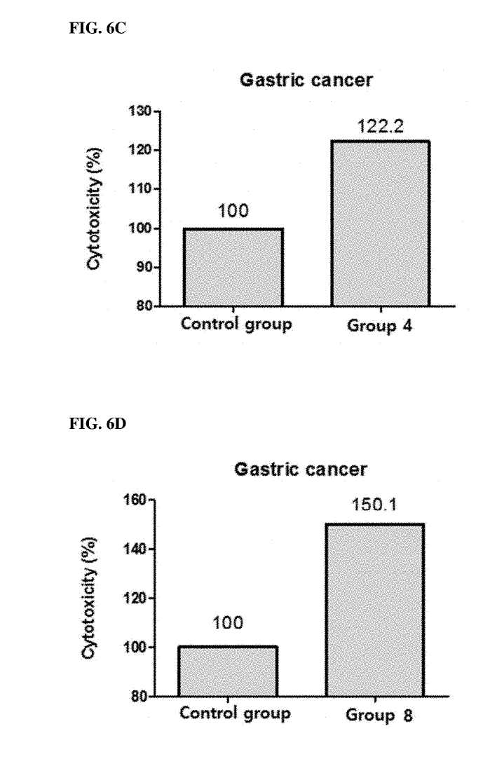

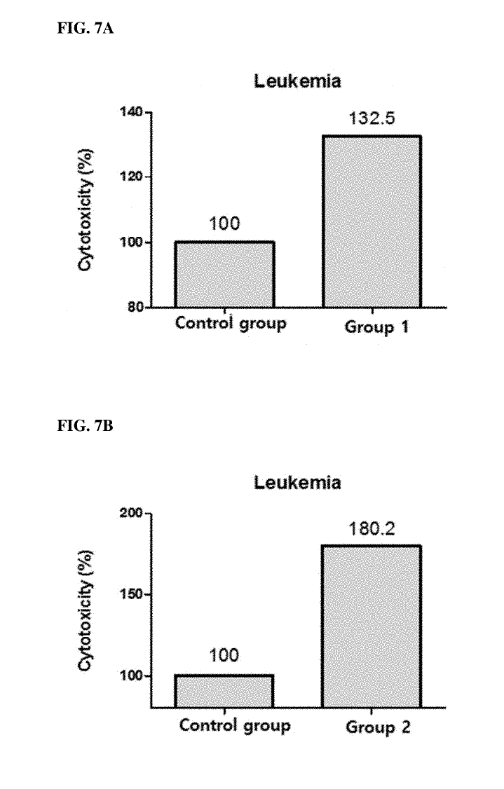

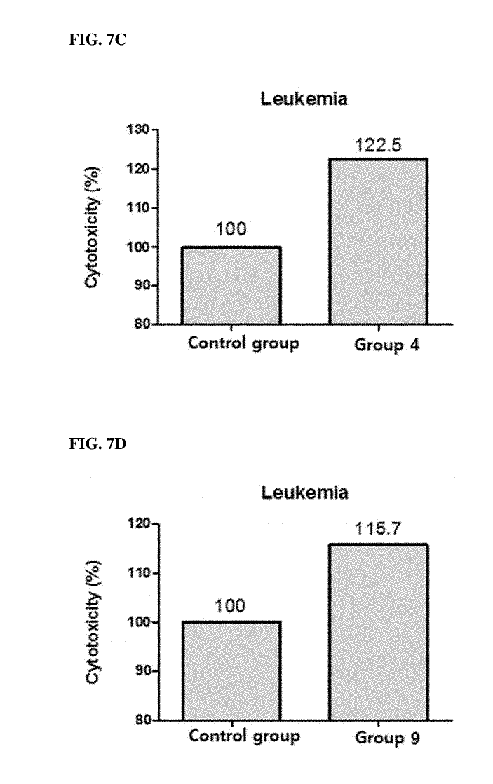

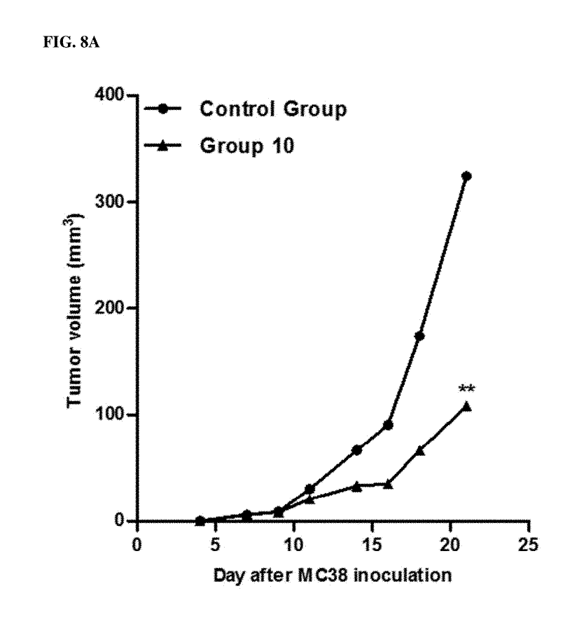

FIGS. 4A, 4B, 4C and 4D provide the results on the colon cancer cell line HCT-116, FIGS. 5A, 5B, 5C and 5D provide the results on the breast cancer cell line MDA-MB-231, FIGS. 6A, 6B, 6C and 6D provide the results on the gastric cancer cell line MKN-74, and FIGS. 7A, 7B, 7C and 7D provide the results on the leukemia cell line U937. As shown in FIGS. 4A to 7D, the results of increasing the cytotoxicity of PBMC when KIRREL2 was neutralized by antibodies or siRNAs were also confirmed in colon cancer, breast cancer, gastric cancer and leukemia.

### Example 3. Tumor-Mouse Model Experiment

This example is to confirm whether the growth of tumor in mouse is suppressed when KIRREL2 is neutralized using KIRREL2 inhibitors.

### 3.1. Establishment of Tumor-Mouse Model

MC-38 cell line derived from C57BL6 colon adenocarcinoma cells was resuspended in 50 μl PBS at the number of 2×10⁵ cells, and was subcutaneously injected into the flanks of 6-week-old female C57BL6 mice.

Table 3 below provides the non-treated control group and Groups 10 and 11 using two siRNAs for knockdown of KIRREL2.

TABLE 3

| | mouse KIRREL2 siRNA |
|---|---|
| Control | Not treated |
| group | |
| Group 10 | Sense (5′-CUCAUGUGUGAAUCCAUCUtt-3′) |
| | (SEQ ID NO: 7) |
| | Antisense (5′-AGAUGGAUUCACACAUGAGtt-3′) |
| | (SEQ ID NO: 8) |
| Group 11 | Sense (5′-CCACCUCUCUCCUUAUGGUtt-3′) |
| | (SEQ ID NO: 9) |
| | Antisense (5′-ACCAUAAGGAGAGAGGUGGtt-3′) |
| | (SEQ ID NO: 10) |

In Groups 10 and 11, the siRNA targeting mouse KIRREL2 was injected into the tumor of mice three times at the interval of 5 days from the 11th day after injecting MC-38 cells. Specifically, 10 μg siRNA and 7.5 μl oligofectamine (Invitrogen) in PBS were mixed according to manufacturer's instruction, and then injected into the tumor tissue induced in mice at a dose of 0.5 mg/kg.

### 3.2. Results

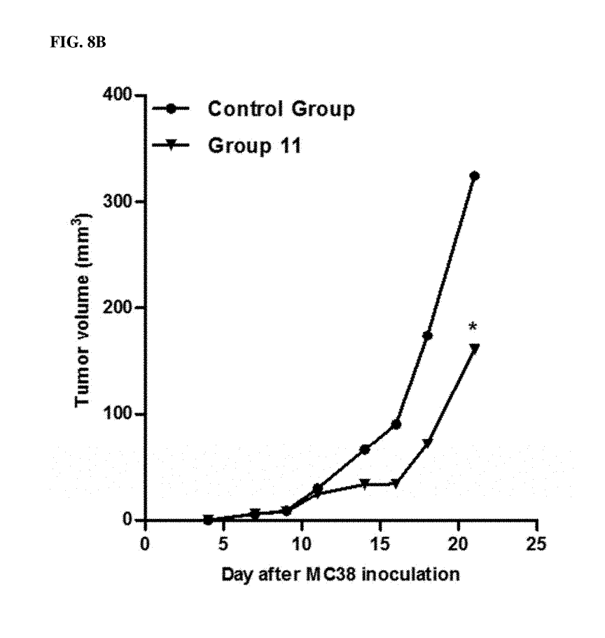

FIGS. 8A and 8B provide the results on the size of tumor induced in mouse of the non-treated control group and Groups 10 and 11.

In the non-treated control group, the tumor continued to grow after it occurred. On the contrary, the growth rate of tumor in mouse was remarkably inhibited in both Groups 10 and 11 compared to the non-treated control group. It means that when KIRREL2 is blocked or knocked down to inhibit its activity or expression, the development of cancer is delayed or stopped and the occurrence of cancer is inhibited. Accordingly, KIRREL2 inhibitor can be efficiently used to prevent cancer.

Those skilled in the art will recognize, or be able to ascertain using no more than routine experimentation, many equivalents to the specific embodiments of the present disclosure described herein. Such equivalents are intended to be encompassed by the following claims.

What is claimed is:

1. A method of treating or preventing cancer, comprising administering a KIRREL2 inhibitor to a subject in need thereof.

2. The method according to claim 1, wherein the KIRREL2 inhibitor is an antisense nucleic acid, a siRNA, a shRNA, a miRNA or a ribozyme that binds in a complementary manner to a DNA or a mRNA of KIRREL2 gene.

3. The method according to claim 1, wherein the KIRREL2 inhibitor is a compound, a peptide, a peptide mimetic, a fusion protein, an antibody or an aptamer that binds specifically to KIRREL2 protein.

4. The method according to claim 1, wherein the cancer is stomach cancer, lung cancer, liver cancer, colorectal cancer, colon cancer, small intestinal cancer, pancreatic cancer, brain cancer, bone cancer, melanoma, breast cancer, sclerosing adenosis, uterine cancer, cervical cancer, head and neck cancer, esophageal cancer, thyroid cancer, parathyroid cancer, renal cancer, sarcoma, prostate cancer, urethral cancer, bladder cancer, blood cancer, leukemia, lymphoma, or fibroadenoma.

5. The method according to claim 1, wherein the KIRREL2 inhibitor suppresses a function of cancer cells evading T cells.

6. A method of immune-enhancing, comprising administering a KIRREL2 inhibitor to a subject in need thereof.

7. The method according to claim 6, wherein the KIRREL2 inhibitor inhibits expression or activity of KIRREL2 in the subject to increase a level of T cell-mediated immune response.

8. The method according to claim 6, wherein the subject is in need of prevention, treatment or improvement of diseases related to immunodeficiency, lower immune function, immune system damage, or immunocompromising.

9. A method of screening an anti-cancer agent comprising:
(a) treating a cancer cell with a candidate anti-cancer agent; and
(b) measuring expression or activity of KIRREL2 in the cancer cell.

10. The method according to claim 9, wherein the step (b) is conducted by determining a level of expression of a mRNA or a protein of KIRREL2 or a level of suppression of T cell activity by KIRREL2.

11. The method according to claim 9 further comprising:
(c) determining the candidate anti-cancer agent to be the anti-cancer agent if a group treated with the candidate anti-cancer agent shows
a significantly lower level of expression of KIRREL2 mRNA or KIRREL2 protein; or
a significantly lower level of suppression of T cell activity by KIRREL2
compared to a group not treated with the candidate anti-cancer agent.

12. A method of providing information necessary for analysis of cancer prognosis comprising: measuring expression or activity of KIRREL2 in cells or tissues isolated from a subject.

13. The method according to claim 12, wherein the expression or activity of KIRREL2 is measured by determining a level of expression of a mRNA or a protein of KIRREL2 or a level of suppression of T cell activity by KIRREL2.


SEQUENCE LISTING (10 sequences)

SEQ ID NO: 1 - DNA, 21 bp, Artificial Sequence
  siRNA against KIRREL2
cgugugacaucuuuccaautt

SEQ ID NO: 2 - DNA, 21 bp, Artificial Sequence
  siRNA against KIRREL2
auuggaaagaugucacacgtt

SEQ ID NO: 3 - DNA, 21 bp, Artificial Sequence
  siRNA against KIRREL2
ccaaccaacgguuacuacatt

SEQ ID NO: 4 - DNA, 21 bp, Artificial Sequence
  siRNA against KIRREL2
uguaguaaccguugguugggt

SEQ ID NO: 5 - DNA, 21 bp, Artificial Sequence
  siRNA against KIRREL2
gagagcaccuuaacccugatt

SEQ ID NO: 6 - DNA, 21 bp, Artificial Sequence
  siRNA against KIRREL2
ucaggguuaaggugcucucca

SEQ ID NO: 7 - DNA, 21 bp, Artificial Sequence
  siRNA against KIRREL2
cucaugugugaauccaucutt

SEQ ID NO: 8 - DNA, 21 bp, Artificial Sequence
  siRNA against KIRREL2
agauggauucacacaugagtt

SEQ ID NO: 9 - DNA, 21 bp, Artificial Sequence
  siRNA against KIRREL2
ccaccucucuccuuauggutt

SEQ ID NO: 10 - DNA, 21 bp, Artificial Sequence
  siRNA against KIRREL2
accauaaggagagagguggtt